(19) United States
(12) Patent Application Publication
(10) Pub. No.: US 2006/0189619 A1
(43) Pub. Date: Aug. 24, 2006

(54) 3-({4-[2-(4-Tert-butylphenyl)-1h-benzimidazol-4-yl]piperazin-1-yl}methyl)pyrido[2,3-b]]pyrazi ne compounds

(75) Inventors: Abdolsamad Tadayon, Kirkland (CA); Jacqueline Lunetta, Pierrefonds (CA); Kiomars Karami, Irvine, CA (US); Zheng Wang, East Brunswick, NJ (US)

Correspondence Address: WILMER CUTLER PICKERING HALE AND DORR LLP /, 60 STATE STREET, BOSTON, MA, 02109, US

(73) Assignee: WYETH, Madison, NJ (US)

(21) Appl. No.: 11/362,468

(22) Filed: Feb. 24, 2006

Related U.S. Application Data
(60) Provisional application No. 60/656,067, filed on Feb. 24, 2005.

(51) Int. Cl. A61K 31/498; C07D 487/02

(52) U.S. Cl. 514/249; 544/330

(57) ABSTRACT

The present invention relates to Gonadotropin Releasing Hormone (“GnRH”) (also known as Leutinizing Hormone Releasing Hormone) receptor antagonists.

This application claims the benefit of provisional application U.S. Serial No. 60/656,067, filed Feb. 24, 2005, which is hereby incorporated by reference into the subject application in its entirety.

This patent disclosure contains material that is subject to copyright protection. The copyright owner has no objection to the facsimile reproduction by anyone of the patent document or the patent disclosure, as it appears in the U.S. Patent and Trademark Office patent file or records, but otherwise reserves any and all copyright rights whatsoever.

### FIELD OF INVENTION

The present invention relates to 3-({4-[2-(4-tert-butylphenyl)-1h-benzimidazol-4-yl]piperazin-1-yl}methyl)pyrido[2,3-b]pyrazine compounds, and their use as Gonadotropin Releasing Hormone (“GnRH”) (also known as Leutinizing Hormone Releasing Hormone) receptor antagonists.

### BACKGROUND

GnRH is a decameric peptide released from the hypothalamus. In the anterior pituitary gland, GnRH activates the GnRH receptor. Activation of the GnRH receptor triggers the release of follicle stimulating hormone (FSH) and leuteinizing hormone (LH). FSH and LH stimulate the biosynthesis and release of sex steroids in the gonads of both genders.

Typically, this is desirable, but certain sex hormone dependent pathological conditions exist where it would be beneficial to prevent activation of the GnRH receptor. For example, inhibition of the GnRH receptor can lead to a large drop in sex steroid production, which in turn can alleviate sex hormone dependent pathological conditions such as prostate cancer, endometriosis, uterine fibroids, uterine cancer, breast cancer, ovarian cancer, testicular cancer, or primary hirsutism. Moreover, there are other situations where it would be beneficial to prevent activation of the GnRH receptor, such as during some points of the in vitro fertilization process, such as to prevent LH surge.

All currently marketed GnRH therapeutics are peptides that exhibit receptor antagonism in one of two ways. The first is through GnRH receptor superagonism. The GnRH receptor, when stimulated in bursts, causes normal release of the gonadotropins, FSH and LH. Under constant stimulation, the receptor becomes desensitized and the overall effect is GnRH receptor inhibition. The superagonism process is somewhat undesirable, as inhibition via this process can take up to two weeks to arise in human patients. During this delay there is often an increase in disease symptoms due to the initial hormone stimulation phase. This phenomenon is referred to as flare.

The second method for receptor inhibition is through direct antagonism of the GnRH receptor with peptide antagonists. This causes an immediate drop in plasma LH levels. However, as mentioned above, current pharmaceuticals that cause blockade of the GnRH receptor are all peptides. As such they are not orally bioavailable and must be administered via parenteral means such as intravenous, subcutaneous or intramuscular injection. Thus, an orally effective GnRH antagonist would be of significant benefit.

Therefore, based upon the foregoing, it is clear that GnRH receptor antagonists are useful, and development of new GnRH receptor antagonists is highly desirable.

### SUMMARY

The present invention relates to 3-({4-[2-(4-tert-butylphenyl)-1H-benzimidazol-4-yl]piperazin-1-yl}methyl)pyrido[2,3-b]pyrazine, and various forms of the same, as well as methods for their use.

### BRIEF DESCRIPTION OF THE DRAWINGS

FIG. 1A is an X-ray diffraction (XRD) scan of the amorphous form of 3-({4-[2-(4-tert-butylphenyl)-1H-benzimidazol-4-yl]piperazin-1-yl}methyl)pyrido[2,3-b]pyrazine.

FIG. 1B is a differential scanning calorimetry (DSC) scan of the amorphous form of 3-({4-[2-4-tert-butylphenyl)-1H-benzimidazol-4-yl]piperazin-1-yl}methyl)pyrido[2,3-b]pyrazine.

FIG. 2A is a thermogravimetric analysis and differential thermal analysis (TGA/DTA) scan of the ethanolate form of 3-({4-[2-(4-tert-butylphenyl)-1H-benzimidazol-4-yl]piperazin-1-yl}methyl)pyrido[2,3-b]pyrazine.

FIG. 2B is an XRD scan of the ethanolate form of 3-(}4-[2-(4-tert-butylphenyl)-1H-benzimidazol-4-yl]piperazin-1-yl)methyl)pyrido[2,3-b]pyrazine.

FIG. 2C is a DSC scan of the ethanolate form of 3-({4-[2-(4-tert-butylphenyl)-1H-benzimidazol-4-yl]piperazin-1-yl}methyl)pyrido[2,3-b]pyrazine.

FIG. 3 is an XRD scan of the hydrate form of 3-({4-[2-(4-tert-butylphenyl)-1H-benzimidazol-4-yl]piperazin-1-yl}methyl)pyrido[2,3-b]pyrazine.

FIG. 4 is a TGA/DTA scan of the hydrate form of 3-({4-[2-(4-tert-butylphenyl)-1H-benzimidazol-4-yl]piperazin-1-yl)methyl)pyrido[2,3-b]pyrazine.

### DETAILED DESCRIPTION

In one embodiment, the present invention comprises 3-({4-[2-(4-tert-butylphenyl)-1H-benzimidazol-4-yl]piperazin-1-yl}methyl)pyrido[2,3-b]pyrazine. The compound is represented by the following structure:

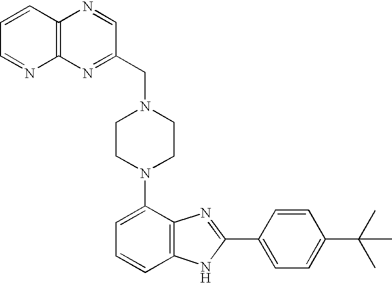

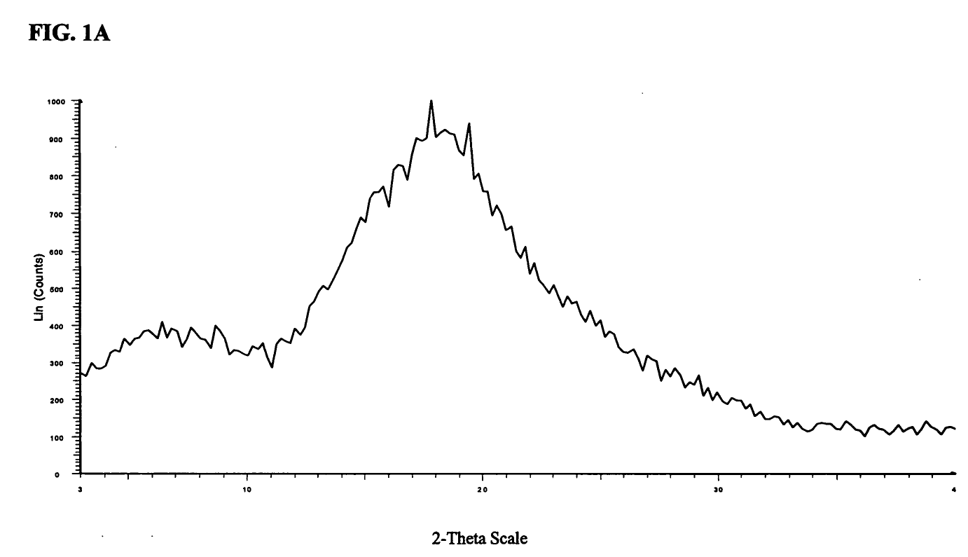

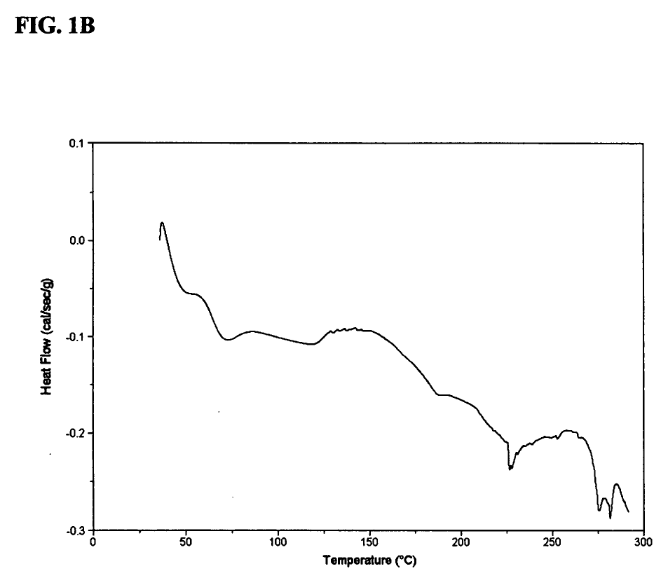

In one embodiment, the compound is the amorphous form of 3-({4-[2-(4-tert-butylphenyl)-1H-benzimidazol-4-yl]piperazin-1-yl}methyl)pyrido[2,3-b]pyrazine. FIGS. 1A-1B provide RD and DSC scans for the amorphous form.

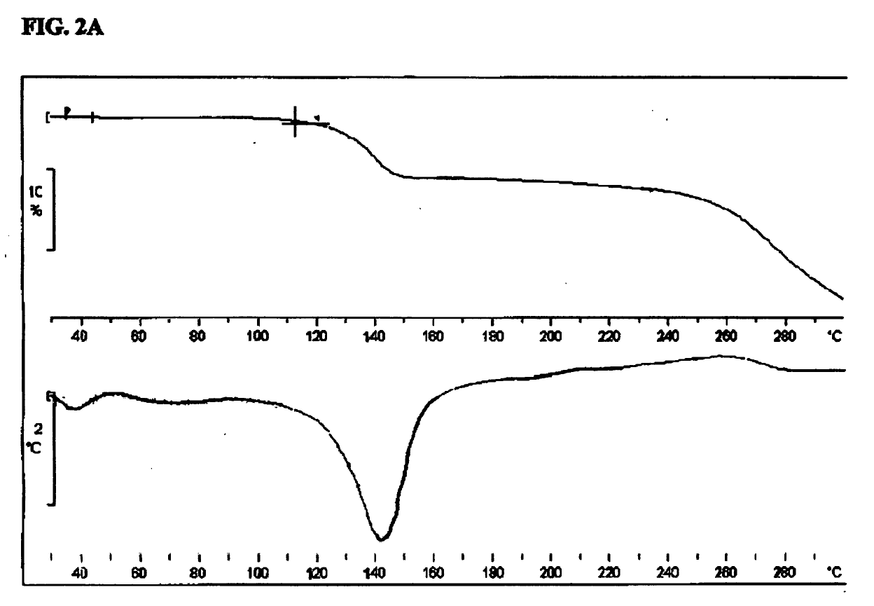

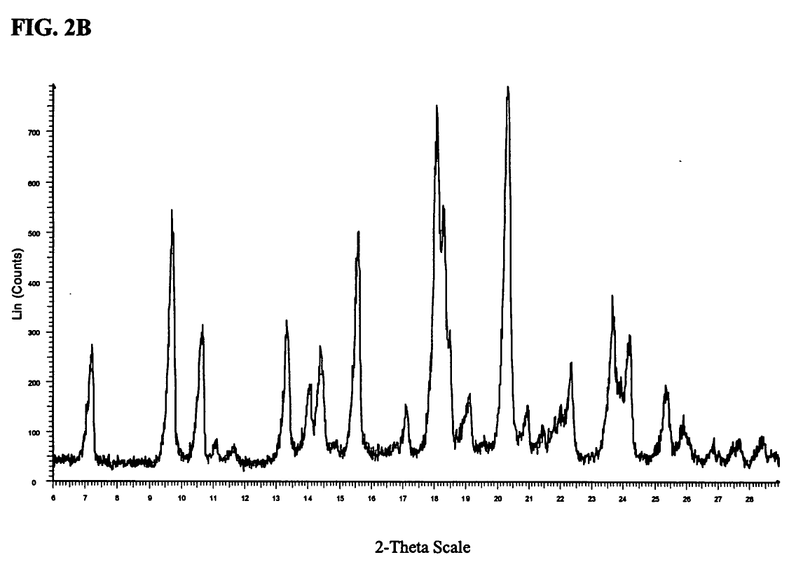

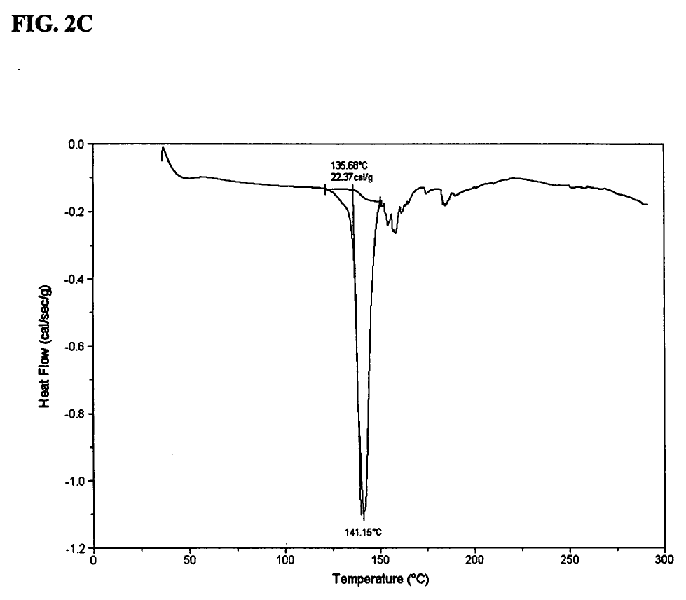

Referring now to FIGS. 2A-2C, in another embodiment, the compound is an ethanolate of 3-({4-[2-(4-tert-butylphenyl)-1H-benzimidazol-4-yl]piperazin-1-yl}methyl)pyrido[2,3-b]pyrazine. The ethanolate form is useful, both as a pharmaceutical composition, and as an intermediate for developing a hydrate form, as will be discussed. The ethanolate form is a mono-ethanolate, and the ethanolate form is crystalline, having an endotherm at about 141° C. The location of the DSC peak may be slightly shifted depending o the particle size distribution, the type of the DSC machine, and the heating rate. A shift of minus/plus 3 degrees is expected. The DSC heating rate was 20° C./min.

Referring to FIG. 2B, the ethanolate form has an X-ray diffraction pattern having peaks expressed in degrees 2θ as disclosed in TABLE 1. The relative intensities of the peaks can vary depending on the sample preparation technique and crystal size distribution, the sample mounting procedure and the particular instrument employed. Moreover, some new peaks may be observed or some existing peaks may be missed depending on the type of machine or the settings (for example whether a Ni filter is used or not). The peaks were collected using a Brukers D8 Advance XRD instrument with no Ni filter used.

TABLE 1

| Angle (2θ) | Intensity (%) |
|---|---|
| 7.179 | 32.8 |
| 9.701 | 66.0 |
| 10.633 | 36.0 |
| 11.089 | 11.1 |
| 11.621 | 9.5 |
| 13.329 | 36.7 |
| 14.039 | 25.4 |
| 14.413 | 31.7 |
| 14.864 | 10.8 |
| 15.574 | 61.3 |
| 17.132 | 20.0 |
| 18.100 | 95.9 |
| 18.300 | 67.1 |
| 19.109 | 21.9 |
| 20.360 | 100.0 |
| 20.939 | 18.4 |
| 21.459 | 12.4 |
| 22.344 | 29.0 |
| 23.708 | 42.2 |
| 24.197 | 37.0 |
| 25.413 | 26.1 |
| 25.946 | 14.4 |
| 26.887 | 9.0 |
| 27.581 | 8.4 |
| 27.737 | 10.1 |
| 28.423 | 12.1 |
| 29.566 | 7.2 |
| 30.811 | 6.3 |
| 32.909 | 6.7 |

The ethanolate has an X-ray diffraction pattern having characteristic peaks expressed in degrees 2θ at 9.701, 18.100, and 20.360. In one embodiment, the ethanolate form has an X-ray diffraction pattern substantially the same as that shown in FIG. 2B.

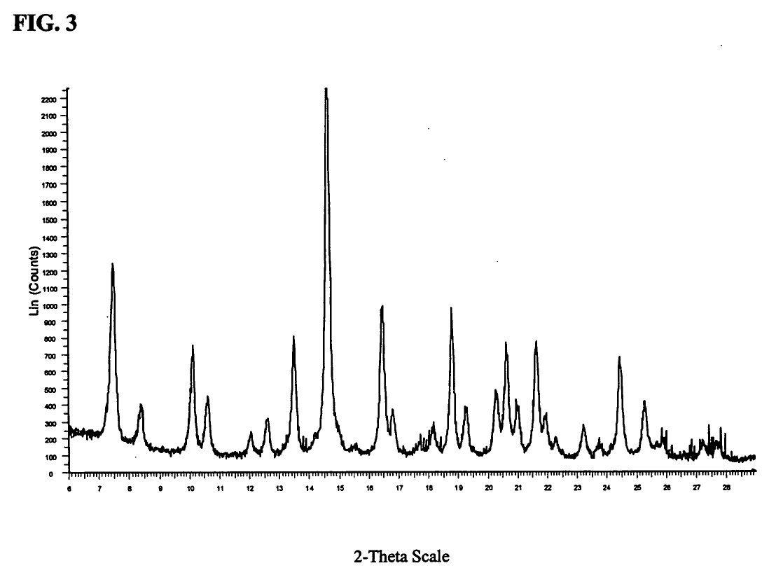

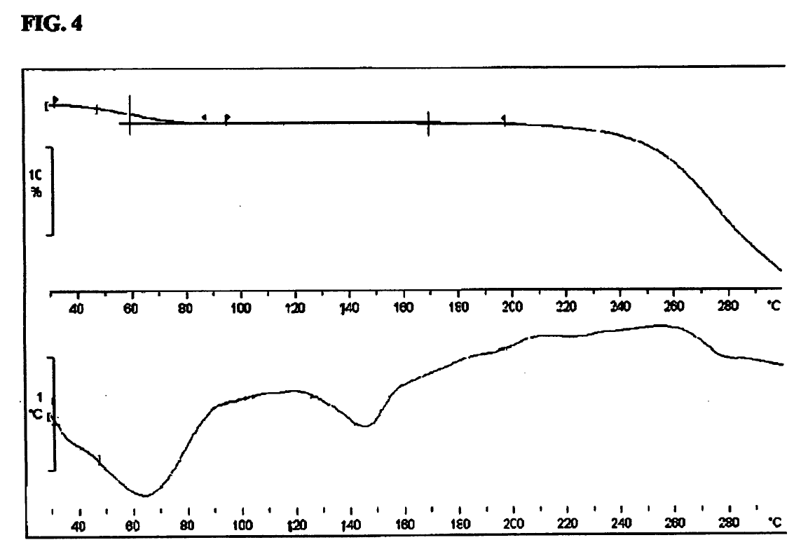

Referring to FIGS. 3-4, in another embodiment, the compound is a hydrate of 3-({4-[2-(4-tert-butylphenyl)-1H-benzimidazol-4-yl]piperazin-1-yl}methyl)pyrido[2,3-b]pyrazine. The hydrate form is a mono-hydrate, and the hydrate form is crystalline, having an endotherm at about 141° C.

Referring to FIG. 3, the hydrate form has an X-ray diffraction pattern having peaks expressed in degrees 2θ as disclosed in TABLE 2. The peaks were collected using a Brukers D8 Advance XRD instrument with no filter used.

TABLE 2

| Angle (2θ) | Intensity (%) |
|---|---|
| 7.449 | 54.5 |
| 8.367 | 17.8 |
| 10.092 | 33.2 |
| 10.585 | 19.6 |
| 12.017 | 9.7 |
| 12.588 | 13.6 |
| 13.482 | 35.2 |
| 14.614 | 100.0 |
| 15.489 | 7.0 |
| 16.442 | 42.7 |
| 16.794 | 16.1 |
| 18.147 | 12.8 |
| 18.780 | 42.6 |
| 19.249 | 16.4 |
| 20.267 | 21.3 |
| 20.622 | 33.5 |
| 20.986 | 17.1 |
| 21.619 | 33.7 |
| 21.920 | 14.3 |
| 22.263 | 9.0 |
| 23.221 | 11.7 |
| 23.738 | 8.5 |
| 24.454 | 28.0 |
| 25.267 | 18.3 |

The hydrate form has an X-ray diffraction pattern having characteristic peaks expressed in degrees 2θ at 7.449, 14.614, 16.442, and 18.780. In one embodiment, the hydrate form has an X-ray diffraction pattern substantially the same as that shown in FIG. 3.

Referring to FIG. 4, the hydrate forms a TGA pattern substantially similar to the Figure. Depending on the drying conditions, normally a 2%-4% weight loss is observed before 130° C.

In one embodiment, the present invention provides a method for modulating the activity of a Gonadotropin Releasing Hormone receptor, comprising contacting said receptor with an effective amount of at least one of the amorphous, ethanolate, and hydrate forms of 3-({4-[2-(4-tert-butylphenyl)-1H-benzimidazol-4-yl]piperazin-1-yl}methyl)pyrido[2,3-b]pyrazine. The method further comprises determining the activity of said receptor. In one embodiment, said determination is made before said contacting step. In another embodiment, said determination is made after said contacting step.

In another embodiment of the present invention, a method for modulating the activity of a Gonadotropin Releasing Hormone (GnRH) receptor, comprising contacting said receptor with an effective amount of at least one of the amorphous, ethanolate, and hydrate forms of 3-({4-[2-(4-tert-butylphenyl)-1H-benzimidazol-4-yl]piperazin-1-yl}methyl)pyrido[2,3-b]pyrazine is provided. In one embodiment, the method further comprises determining the activity of said receptor. Further, in one embodiment, the determination is made before said contacting step. In another embodiment, the determination is made after said contacting step.

In another embodiment of the present invention, a method for treating a patient suspected of suffering from a condition associated with excessive Gonadotropin Releasing Hormone (GnRH) receptor activity, comprising the step of administering to the patient a therapeutically effective amount of at least one of the amorphous, ethanolate, and hydrate forms of 3-({4-[2-(4-tert-butylphenyl)-1H-benzimidazol-4-yl]piperazin-1-yl}methyl)pyrido[2,3-b]pyrazine is provided. In one embodiment, the condition is prostate cancer, endometriosis, uterine fibroids, uterine cancer, breast cancer, ovarian cancer, testicular cancer, primary hirsutism, or LH surge.

In one embodiment, the compounds of the present invention are administered in combination with an additional active agent. Preferably, the additional active agent is selected from the group consisting of at least one of androgens, estrogens, progesterones, antiestrogens, antiprogestogens, testosterone, antiprogestogens, angiotensin-converting enzyme inhibitor (such as ENALAPRIL or CAPTOPRIL), angiotensin II-receptor antagonist (such as LOSARTAN), renin inhibitor, bisphosphonates (bisphosphonic acids), growth hormone secretagogues (such as MK-0677), 5a-reductase 2 inhibitor (such as finasteride or epristeride), a 5a-reductase 1 inhibitor (such as 4,7b-dimethyl-4-aza-5a-cholestan-3-one, 3-oxo-4-aza-4,7b-dimethyl-16b-(4-chlorophenoxy)-5a-androstane, and 3-oxo-aza-4,7b-dimethyl-16b-(phenoxy)-5a-androstane), dual inhibitors of 5a-reductase 1 and 5a-reductase 2 (such as 3-oxo-4-aza-17b-(2,5-trifluoromethylphenyl-carbamoyl)-5a-androstan), antiandrogens (such as flutamide, casodex and cyproterone acetate), alpha-1 blockers (such as prazosin, terazosin, doxazosin, tamsulosin, and alfuzosin), growth hormone, and luteinizing hormone releasing compounds (such as a peptide (including leuprorelin, gonadorelin, buserelin, triptorelin, goserelin, nafarelin, histrelin, deslorelin, meterlin and recirelin) or natural hormone or analog thereof). For example, when used with compounds of the present invention: androgens, estrogens, progesterones, antiestrogens and antiprogestogens find use in the treatment of endometriosis, fibroids and in contraception; testosterone or other androgens or antiprogestogens find use in men as a contraceptive; angiotensin-converting enzyme inhibitors, angiotensin II-receptor antagonists, and renin inhibitor find use in the treatment of uterine fibroids; bisphosphonates (bisphosphonic acids) and growth hormone secretagogues find use in the treatment and prevention of disturbances of calcium, phosphate and bone metabolism, in particular, for the prevention of bone loss during therapy with the GnRH antagonist, and in combination with estrogens, progesterones, antiestrogens, antiprogestins and/or androgens for the prevention or treatment of bone loss or hypogonadal symptoms such as hot flashes during therapy with the GnRH antagonist; 5a-reductase 2 inhibitor, 5a-reductase 1 inhibitor, dual inhibitors of 5a-reductase 1 and 5a-reductase 2, antiandrogens, and alpha-1 blockers are useful as well; growth hormone, growth hormone releasing hormone or growth hormone secretagogues, to delay puberty in growth hormone deficient children; a compound having luteinizing hormone releasing activity is useful as well.

In another embodiment, the present invention provides a method for converting amorphous 3-({4-[2-(4-tert-butylphenyl)-1H-benzimidazol4-yl]piperazin-1-yl)methyl)pyrido[2,3-b]pyrazine to a hydrate form, the method comprising dissolving amorphous 3-({4-[2-(4-tert-butylphenyl)-1H-benzimidazol-4-yl]piperazin-1-yl}methyl)pyrido[2,3-b]pyrazine in ethanol; obtaining the ethanolate form of 3-({4-[2-(4-tert-butylphenyl)-1H-benzimidazol-4-yl]piperazin-1-yl}methyl)pyrido[2,3-b]pyrazine; contacting the ethanolate form with water at a temperature above 25° C.; and crystallizing to obtain the hydrate form of 3-({4-[2-(4-tert-butylphenyl)-1H-benzimidazol-4-yl]piperazin-1-yl}methyl)pyrido[2,3-b]pyrazine.

Methods of Making

### Methods of making 3-({4-[2-(4-tert-butylphenyl)-1H-benzImidazol-4-yl]piperazin-1-yl}methyl)pyrido[2,3-b]pyrazine

This application incorporates the disclosures of U.S. Provisional Application Nos. 60/580,640 and 60/580,665, both filed Jun. 17, 2004, by reference in their entireties.

For preparing 3-({4-[2-(4-tert-butylphenyl)-1H-benzimidazol-4-yl]piperazin-1-yl}methyl)pyrido[2,3-b]pyrazine, the key intermediate 4 can be prepared in two ways (Schemes 1 and 2). In scheme 1,2,6-difluoronitrobenzene 1 is treated with a slight excess of sodium azide for 2 hours then the reaction mixture is treated with a 50% excess of piperazine, 2-substituted piperazine or 2,6-disubstituted piperazine in unprotected form or protected at the more hindered nitrogen as a Boc or Cbz function. Intermediate 2 is obtained in yields ranging from 50-90%. The nitro and azide functions are reduced under standard catalytic conditions (H2, Pt/C, MeOH) and the product phenylenediamine is treated with a substituted benzaldehyde and Pd/C to promote oxidation. The product benzimidazole is deprotected if necessary (H₂, Pd/C if PG=Cbz; TFA-DCM if PG Boc) and the product, in most cases, can be crystallized from acetonitrile.

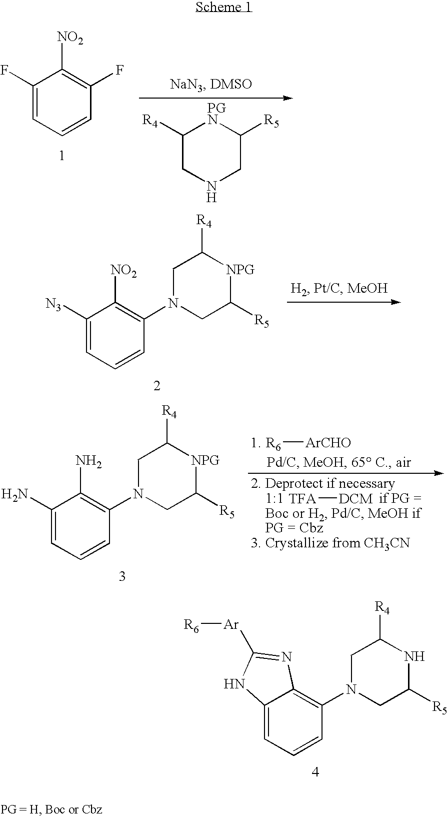

Wherein R₄ and R₅ are H; R₆ is t-butyl; Ar ispara-substituted phenyl.

Scheme 2 indicates that the phenylenediamine intermediate 3 can be condensed with an acid and the product amide can be reacted with weak acid to cyclize and provide the intermediate 4 after deprotection.

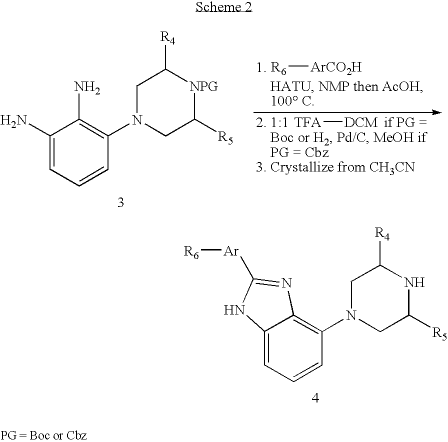

Wherein R₄ and R₅ are H; R₆ is t-butyl; Ar ispara-substituted phenyl.

Substitution on the secondary nitrogen of the piperazine ring is achieved in three ways: Scheme 3 shows substitution occurring through nucleophilic substitution of an alkyl halide to provide the target products (I). 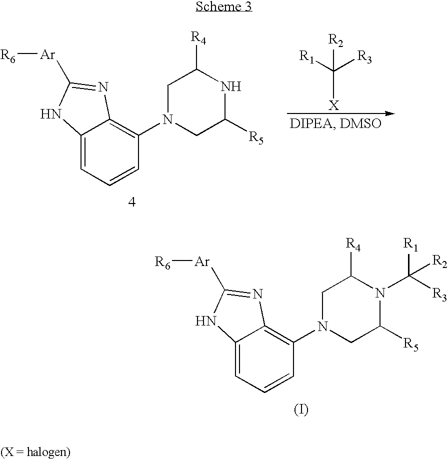
Wherein R₃ is

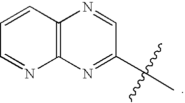

Scheme 4 indicates products (I) are prepared via reductive amination between aldehydes/ketones and the intermediate 4.

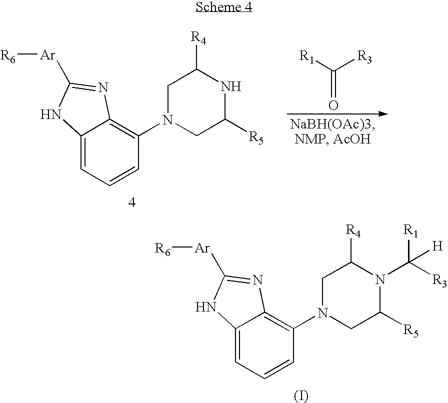

Wherein R₃ is

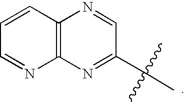

Scheme 5 indicates products () can also be obtained by condensing intermediate 4 with an activated acid to form and amide. The amide can be reduced under certain conditions to provide (I). 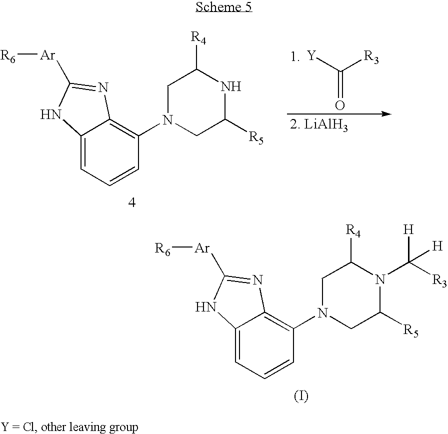
Method of Making the Hydrate

In one embodiment, a procedure for converting the amorphous 3-({4-[2-(4-tert-butylphenyl)-1H-benzimidazol-4-yl]piperazin-1-yl}methyl)pyrido[2,3-b]pyrazine to the hydrate is provided, the method comprising:
Adding 4 volume of ethanol to the starting material, amorphous 3-({4-[2-(4-tert-butylphenyl)-1H-benzimidazol-4-yl]piperazin-1-yl}methyl)pyrido[2,3-b]pyrazine, and stirring at room temperature for about 6 hrs. The solids dissolve and then crystallize. If the solids do not crystallize, adding a small amount of ethanolate seeds.
Filtering the solids, leaving solids on the filter for at least 1 hr (larger batches may require longer time) to make sure that the solids do not contain much residual ethanol. In one embodiment, overnight drying of ethanolate is recommended. Alternatively, it is also possible to use the solids undried.
Adding the ethanolate solids to 10 volumes of water, maintaining jacket temperature at about 55° C. Stirring the suspension for 24 hrs. If the ethanolate solids were not dried (such as in an oven), jacket temperature (during hydration) preferably is maintained at about 30 to about 50° C.
Adding hydrate seeds.
Taking a few small samples during hydration and monitoring the process progress by performing an in-process test for ethanol content (see Example 5). If progress is not noticed after 5 hrs, increase stirring rate.
After complete transformation is confirmed, cooling the solution to room temperature. Filtering the suspension and drying the solids at about about 40 to about 60° C. overnight.
   Definitions

All recitations of a group, such as alkyl, are understood for the purposes of this specification to encompass both substituted and unsubstituted forms.

The term “phenyl”, as used herein, whether used alone or as part of another group, refers to a substituted or unsubstituted phenyl group.

The term “pharmaceutically acceptable salt”, as used herein, refers to salts derived form organic and inorganic acids such as, for example, acetic, propionic, lactic, citric, tartaric, succinic, fumaric, maleic, malonic, mandelic, malic, phthalic, hydrochloric, hydrobromic, phosphoric, nitric, sulfuric, methanesulfonic, napthalenesulfonic, benzenesulfonic, toluenesulfonic, camphorsulfonic, and similarly known acceptable acids when a compound of this invention contains a basic moiety. Salts may also be formed from organic and inorganic bases, preferably alkali metal salts, for example, sodium, lithium, or potassium, when a compound of this invention contains a carboxylate or phenolic moiety, or similar moiety capable of forming base addition salts.

The term “patient”, as used herein, refers to a mammal, preferably a human.

The terms “administer”, “administering”, or “administration”, as used herein, refer to either directly administering a compound or composition to a patient, or administering a prodrug derivative or analog of the compound to the patient, which will form an equivalent amount of the active compound or substance within the patient's body.

The term “carrier”, as used herein, shall encompass carriers, excipients, and diluents.

The compounds of this invention may contain an asymmetric carbon atom and some of the compounds of this invention may contain one or more asymmetric centers and may thus give rise to optical isomers and diastereomers. While shown without respect to stereochemistry in formula I, the present invention includes such optical isomers and diastereomers; as well as the racemic and resolved, enantiomerically pure R and S stereoisomers; as well as other mixtures of the R and S stereoisomers and pharmaceutically acceptable salts thereof. Where a stereoisomer is preferred, it may in some embodiments be provided substantially free of the corresponding enantiomer. Thus, an enantiomer substantially free of the corresponding enantiomer refers to a compound that is isolated or separated via separation techniques or prepared free of the corresponding enantiomer. “Substantially free”, as used herein, means that the compound is made up of a significantly greater proportion of one steriosomer, preferably less than about 50% of the other, more preferably less than about 75%, and even more preferably less than about 90%.

The terms “effective amount”, “therapeutically effective amount” and “effective dosage” as used herein, refer to the amount of a compound, that, when administered to a patient, is effective to at least partially ameliorate (and, in preferred embodiments, cure) a condition form which the patient is suspected to suffer.

Compounds of the present invention have been found to act as GnRH receptor antagonists. They are therefore useful in the treatment of prostate cancer, endometriosis, uterine fibroids, uterine cancer, breast cancer, ovarian cancer, testicular cancer, primary hirsutism, or LH surge. In addition, they are useful as oral contraceptives. The present invention thus provides pharmaceutical compositions comprising at least one compound of the present invention and one or more pharmaceutically acceptable carriers, excipients, or diluents.

Examples of such carriers are well known to those skilled in the art and are prepared in accordance with. acceptable pharmaceutical procedures, such as, for example, those described in Remington's Pharmaceutical Sciences, 17th edition, ed. Alfonoso R. Gennaro, Mack Publishing Company, Easton, Pa. (1985), which is incorporated herein by reference in its entirety. Pharmaceutically acceptable carriers are those that are compatible with the other ingredients in the formulation and biologically acceptable.

The compounds of this invention may be administered orally or parenterally, neat or in combination with conventional pharmaceutical carriers. Applicable solid carriers can include one or more substances which may also act as flavoring agents, lubricants, solubilizers, suspending agents, fillers, glidants, compression aids, binders or tablet-disintegrating agents or encapsulating materials. They are formulated in conventional manner, for example, in a manner similar to that used for known antihypertensive agents, diuretics and β-blocking agents. Oral formulations containing the active compounds of this invention may comprise any conventionally used oral forms, including tablets, capsules, buccal forms, troches, lozenges and oral liquids, suspensions or solutions. In powders, the carrier is a finely divided solid, which is an admixture with the finely divided active ingredient. In tablets, the active ingredient is mixed with a carrier having the necessary compression properties in suitable proportions and compacted in the shape and size desired. The powders and tablets preferably contain up to 99% of the active ingredient.

Capsules may contain mixtures of the active compound(s) with inert fillers and/or diluents such as the pharmaceutically acceptable starches (e.g. corn, potato or tapioca starch), sugars, artificial sweetening agents, powdered celluloses, such as crystalline and microcrystalline celluloses, flours, gelatins, gums, etc.

Useful tablet formulations may be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrrolidine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes and ion exchange resins. Preferred surface modifying agents include nonionic and anionic surface modifying agents. Representative examples of surface modifying agents include, but are not limited to, poloxamer 188, benzalkonium chloride, calcium stearate, cetostearl alcohol, cetomacrogol emulsifying wax, sorbitan esters, colliodol silicon dioxide, phosphates, sodium dodecylsulfate, magnesium aluminum silicate, and triethanolamine. Oral formulations herein may utilize standard delay or time release formulations to alter the absorption of the active compound(s). The oral formulation may also consist of administering the active ingredient in water or fruit juice, containing appropriate solubilizers or emulisifiers as needed.

Liquid carriers may be used in preparing solutions, suspensions, emulsions, syrups and elixirs. The active ingredient of this invention can be dissolved or suspended in a pharmaceutically acceptable liquid carrier such as water, an organic solvent, a mixture of both or pharmaceutically acceptable oils or fat. The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmo-regulators. Suitable examples of liquid carriers for oral and parenteral administration include water (particularly containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration the carrier can also be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellant.

Liquid pharmaceutical compositions, which are sterile solutions or suspensions, can be utilized by, for example, intramuscular, intraperitoneal or subcutaneous injection. Sterile solutions can also be administered intravenously. Compositions for oral administration may be in either liquid or solid form.

Preferably the pharmaceutical composition is in unit dosage form, e.g. as tablets, capsules, powders, solutions, suspensions, emulsions, granules, or suppositories. In such form, the composition is sub-divided in unit dose containing appropriate quantities of the active ingredient; the unit dosage forms can be packaged compositions, for example, packeted powders, vials, ampoules, prefilled syringes or sachets containing liquids. The unit dosage form can be, for example, a capsule or tablet itself, or it can be the appropriate number of any such compositions in package form. Such unit dosage form may contain from about 1 mg/kg to about 250 mg/kg, and may given in a single dose or in two or more divided doses. Such doses may be administered in any manner useful in directing the active compounds herein to the recipient's bloodstream, including orally, via implants, parenterally (including intravenous, intraperitoneal and subcutaneous injections), rectally, vaginally, and transdermally. Such administrations may be carried out using the present compounds, or pharmaceutically acceptable salts thereof, in lotions, creams, foams, patches, suspensions, solutions, and suppositories (rectal and vaginal).

When administered for the treatment or inhibition of a particular disease state or disorder, it is understood that the effective dosage may vary depending upon the particular compound utilized, the mode of administration, the condition, and severity thereof, of the condition being treated, as well as the various physical factors related to the individual being treated. In therapeutic application, compounds of the present invention are provided to a patient already suffering from a disease in an amount sufficient to cure or at least partially ameliorate the symptoms of the disease and its complications. An amount adequate to accomplish this is defined as a “therapeutically effective amount”. The dosage to be used in the treatment of a specific case must be subjectively determined by the attending physician. The variables involved include the specific condition and the size, age and response pattern of the patient.

In some cases it may be desirable to administer the compounds directly to the airways in the form of an aerosol. For administration by intranasal or intrabrochial inhalation, the compounds of this invention may be formulated into an aqueous or partially aqueous solution.

The compounds of this invention may be administered parenterally or intraperitoneally. Solutions or suspensions of these active compounds as a free base or pharmaceutically acceptable salt may be prepared in water suitably mixed with a surfactant such as hydroxyl-propylcellulose. Dispersions may also be prepared in glycerol, liquid polyethylene glycols and mixtures thereof in oils. Under ordinary conditions of storage and use, these preparations contain a preservative to inhibit the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

The compounds of this invention can be administered transdermally through the use of a transdermal patch. For the purposes of this disclosure, thransdermal administrations are understood to include all administrations across the surface of the body and the inner linings of bodily passages including epithelial and mucosal tissues. Such administrations may be carried out using the present compounds, or pharmaceutically acceptable salts thereof, in lotions, creams, foams, patches, suspensions, solutions, and suppositories (rectal and vaginal).

Transdermal administration may be accomplished through the use of a transdermal patch containing the active compound and a carrier that is inert to the active compound, is non-toxic to the skin, and allows delivery of the agent for systemic absorption into the blood stream via the skin. The carrier may take any number of forms such as creams and ointments, pastes, gels and occlusive devices. The creams and ointments may be viscous liquid or semisolid emulsions of either the oil-in-water or water-in-oil type. Pastes comprised of absorptive powders dispersed in petroleum or hydrophilic petroleum containing the active ingredient may also be suitable. A variety of occlusive devices may be used to release the active ingredient into the blood stream, such as a semi-permeable membrane covering a reservoir containing the active ingredient with or without a carrier, or a matrix containing the active ingredient. Other occlusive devices are known in the literature.

The compounds of this invention may be administered rectally or vaginally in the form of a conventional suppository. Suppository formulations may be made from traditional materials, including cocoa butter, with or without the addition of waxes to alter the suppository's melting point, and glycerin. Water soluble suppository bases, such as polyethylene glycols of various molecular weights, may also be used.

In certain embodiments, the present invention is directed to prodrugs of compounds of the present invention. Various forms of prodrugs are known in the art, for example, as discussed in, for example, Bundgaard, (ed.), Design of Prodrugs, Elsevier (1985); Widder, et al. (ed.), Methods in Enzymology, vol. 4, Academic Press (1985); Krogsgaard-Larsen, et al. (ed.), “Design and Application of Prodrugs”, Textbook of Drug Design and Development, Chapter 5, 113-191 (1991), Bundgaard, et al., Journal of Drug Delivery reviews, 8:1-38 (1992), Bundgaard, J. of Pharmaceutical Sciences, 77:285 et seq. (1988); and Higuchi and Stella (eds.) Prodrugs as Novel Drug Delivery Systems, American Chemical Society (1975), each of which is incorporated by reference in its entirety.

It is understood that the dosage, regimen and mode of administration of these compounds will vary according to the malady and the individual being treated and will be subject to the judgment of the medical practitioner involved. It is preferred that the administration of one or more of the compounds herein begin at a low dose and be increased until the desired effects are achieved.

The compounds of the invention can be prepared using a variety of methods starting from commercially available compounds, known compounds, or compounds prepared by known methods. General synthetic routes to many of the compounds of the invention are included in the following schemes. It is understood by those skilled in the art that protection and deprotection steps not shown in the Schemes may be required for these syntheses, and that the order of steps may be changed to accommodate functionality in the target molecules.

The present compounds are further described in the following examples.

### EXAMPLES

### Example 1

### Forming The Ethanolate And Its General Physical Properties

Referring to FIGS. 1-2, 0.8 g of 3-({4-[2-(4-tert-butylphenyl)-1H-benzimidazol-4-yl]piperazin-1-yl}methyl)pyrido[2,3-b]pyrazine characterized by DSC and TGA/DTA scans was added to 4 volumes (3.2 ml) of ethanol and stirred at room temperature for 6 hrs. The solid first dissolved and then precipitated from a clear solution. The resulting precipitate was dried at 80° C. and full vacuum overnight. 100701 The precipitate was believed to be the ethanolate form of 3-({4-[2-(4-tert-butylphenyl)-1H-benzimidazol-4-yl]piperazin-1-yl ) methyl)pyrido[2,3-b]pyrazine. Referring to FIG. 2A, TGA showed 7 wt % solvent content. The ethanol content in the dry solid as measured by GC was 9 wt %. Theoretical ethanol content for a mono-ethanolate is 8.8%.

Referring to FIG. 2B, XRD showed that the dry solid is crystalline.

Referring to FIG. 2C, DSC showed that there is sharp endotherm with onset at 136° C. (apex at 141° C.). Hot stage microscopy showed melting occurs at around 149° C. Based on this information, the compound is a monoethanolate with melting and desolavation occurring approximately at the same temperature. This means that the ethanol molecule is detached from the compound when crystalline structure is lost (showing that the ethanol is strongly attached to the lattice structure). The process yield is approximately 87 wt %.

### Example 2

### Purification

50 mg crude of the amorphous form of 3-({4-[2-(4-tert-butylphenyl)-1H-benzimidazol-4-yl]piperazin-1-yl}methyl)pyrido[2,3-b]pyrazine was added to 0.6 ml ethanol at room temperature for 2 hrs. The solids first dissolved and then crystallized. The materials were filtered but not washed with ethanol. HPLC analysis showed that the purity of the of the amorphous form of 3-({4-[2-(4-tert-butylphenyl)-1H-benzimidazol-4-yl]piperazin-1-yl}methyl)pyrido[2,3-b]pyrazine increased from around 90% to 97%. Washing with ethanol on the filter may further reduce the impurity level. Thus, it was discovered that the method of Example 1 also purifies the crude starting material.

### Example 3

### Selenium Content

1.00 gr of the amorphous form of 3-({4-[2-(4-tert-butylphenyl)-1H-benzimidazol-4-yl]piperazin-1-yl}methyl)pyrido[2,3-b]pyrazine was added to 4 volumes of ethanol at room temperature, stirred for 5 hrs. The solids were dried in the oven at 80° C. overnight. The Se content reduced from 110 to 60 ppm. TGA indicated 8% ethanol content. A similar experiment showed that Se content decreased from 110 to 85 ppm. Thus, it was discovered that the method of Example I also partially removes selenium present in the crude starting material.

### Example 4

### Solvent Exchange: Forming A Hydrate By Removing Ethanol And Hydrating 3-({4-12-(4-tert-butylphenyl)-1H-benzimidazol-4-yl]piperazin-1-yl)methyl)pyrido[2,3-blpyrazine

152 mg of solids obtained from Example 1 were added to 1.5 ml water at 70° C. The suspension was stirred for approximately 15 h; a sample was taken after 3 hrs stirring and a second sample was taken at the end of the experiment. Both samples were dried at 80° C. overnight. The first sample showed 2.7% weight loss before 100° C., the second sample showed 2.4% weight loss in the same temperature range on TGA (temperature ramp=20° C./min). Sample analysis showed that the second sample contained 2% water and 0.1% ethanol. The weight loss below 110° C. is an indication of water removal. The weight loss around 140° C. is an indication of ethanol removal. In both cases a hydrate was formed (an ethanol molecule was removed from the molecule). A similar experiment showed that overnight stirring at room temperature decreases the ethanol content from 8% to 4.4%.

### Example 5

### In-Process Test During Hydration Of The Compound

When the ethanolate is reslurried in water, a TGA test may be used to check the ethanol content of the solids. The weight drop between 135° C. to 195° C. on the TGA scan is approximately equal to the ethanol content. The heating rate for TGA may be set at 20° C./min. A 50 mg solids sample dried in the oven for 15 min at 60° C. under vacuum is sufficiently dried to be used for the TGA test.

The disclosures of each patent, patent application, and publication cited or described in this document are hereby incorporated herein by reference, in their entireties.

Various modifications of the invention, in addition to those described herein, will be appparent to those skilled in the art from the foregoing description. Such modifications are also intended to fall within the scope of the appended claims.

What is claimed:

1. A compound which is the amorphous, ethanolate, or hydrate form of 3-({4-[2-(4-tert-butylphenyl)-1H-benzimidazol-4-yl]piperazin-1-yl}methyl)pyrido[2,3-b]pyrazine.

2. The compound of claim 1, wherein the compound is the amorphous form of 3-({4-[2-(4-tert-butylphenyl)-1H-benzimidazol-4-yl]piperazin-1-yl}methyl)pyrido[2,3-b]pyrazine.

3. The compound of claim 1, wherein the compound is the ethanolate form of 3-({4-[2-(4-tert-butylphenyl)-1H-benzimidazol-4-yl]piperazin-1-yl}methyl)pyrido[2,3-b]pyrazine.

4. The compound of claim 3, wherein the compound is 3-({4-[2-(4-tert-butylphenyl)-1H-benzimidazol-4-yl]piperazin-1-yl}methyl)pyrido[2,3-b]pyrazine mono-ethanolate.

5. The compound of claim 3, wherein the ethanolate is crystalline.

6. The compound of claim 5, having an endotherm at about 141° C. on DSC at 10° C./min heating rate.

7. The compound of claim 5, having an X-ray diffraction pattern having characteristic peaks expressed in degrees 20 at 9.701, 18.100, and 20.360.

8. The compound of claim 5, having an X-ray diffraction pattern substantially the same as that shown in FIG. 2B.

9. The compound of claim 1, wherein the compound is a hydrate of 3-({4-[2-(4-tert-butylphenyl)-1H-benzimidazol-4-yl]piperazin-1-yl}methyl)pyrido[2,3-b]pyrazine.

10. The compound of claim 9, wherein the compound is a mono-hydrate.

11. The compound of claim 9, wherein the compound is crystalline.

12. The compound of claim 11, having an endotherm at about 141° C. on DSC at 10° C./min heating rate.

13. The compound of claim 11, having an X-ray diffraction pattern having characteristic peaks expressed in degrees 20 at 7.449, 14.614, 16.442, and 18.780.

14. The compound of claim 11, having an X-ray diffraction pattern substantially the same as that shown in FIG. 3.

15. A method for modulating the activity of a Gonadotropin Releasing Hormone receptor, comprising contacting said receptor with an effective amount of a compound of claim 1.

16. The method of claim 15, wherein the compound is a hydrate of 3-({4-[2-(4-tert-butylphenyl)-1H-benzimidazol-4-yl]piperazin-1-yl}methyl)pyrido[2,3-b]pyrazine.

17. The method of claim 15, further comprising determining the activity of said receptor.

18. The method of claim 17, wherein said determination is made before said contacting step.

19. The method of claim 17, wherein said determination is made after said contacting step.

20. A method for treating a patient suspected of suffering from a condition associated with excessive Gonadotropin Releasing Hormone receptor activity, comprising administering to the patient a therapeutically effective amount of a compound according to claim 1.

21. The method of claim 20, wherein the compound is a hydrate of 3-({4-[2-(4-tert-butylphenyl)-1H-benzimidazol-4-yl]piperazin-1-yl}methyl)pyrido[2,3-b]pyrazine.

22. The method of claim 20, wherein said condition is prostate cancer, endometriosis, uterine fibroids, uterine cancer, breast cancer, ovarian cancer, testicular cancer, primary hirsutism, or LH surge.

23. A pharmaceutical composition, comprising:
a compound according to claim 1; and
an additional active agent selected from the group consisting of at least one of androgens, estrogens, progesterones, antiestrogens, antiprogestogens, testosterone, antiprogestogens, angiotensin-converting enzyme inhibitor, angiotensin II-receptor antagonist, renin inhibitor, bisphosphonates, growth hormone secretagogues, 5a-reductase 2 inhibitor, a 5a-reductase 1 inhibitor, dual inhibitors of 5a-reductase 1 and 5a-reductase 2, antiandrogens, alpha-1 blockers, growth hormone, and luteinizing hormone releasing compounds.

24. The composition of claim 23, wherein the compound is a hydrate of 3-({4-[2-(4-tert-butylphenyl)-1H-benzimidazol-4-yl]piperazin-1-yl}methyl)pyrido[2,3-b]pyrazine.

25. A method for converting amorphous 3-({4-[2-(4-tert-butylphenyl)-1H-benzimidazol-4-yl]piperazin-1-yl}methyl)pyrido[2,3-b]pyrazine to a hydrate form, the method comprising:
dissolving amorphous 3-({4-[2-(4-tert-butylphenyl)-1H-benzimidazol-4-yl]piperazin-1-yl}methyl)pyrido[2,3-b]pyrazine in ethanol; obtaining the ethanolate form of 3-({4-[2-(4-tert-butylphenyl)-1H-benzimidazol-4-yl]piperazin-1-yl} methyl)pyrido[2,3-b]pyrazine;
contacting the ethanolate form with water at a temperature above 25° C.; and
crystallizing to obtain the hydrate form of 3-({4-[2-(4-tert-butylphenyl)-1H-benzimidazol-4-yl]piperazin-1-yl methyl)pyrido[2,3-b]pyrazine.

26. The method of claim 25, wherein crystallizing includes filtering the solids to obtain the hydrate form and drying the solids at about about 40 to about 60° C. overnight.
